(19) United States
(12) Patent Application Publication
(10) Pub. No.: US 2010/0036218 A1
(43) Pub. Date: Feb. 11, 2010

(54) FINGERTIP BLOOD OXYGEN SATURATION MEASURING APPARATUS

(75) Inventors: Weihu Wang, Beijing (CN); Peng Wu, Beijing (CN); Lei Chen, Beijing (CN)

Correspondence Address: SEYFARTH SHAW LLP, WORLD TRADE CENTER EAST, TWO SEAPORT LANE, SUITE 300, BOSTON, MA, 02210-2028, US

(73) Assignee: BEIJING CHOICE ELECTRONIC TECHNOLOGY CO., LTD., Beijing (CN)

(21) Appl. No.: 12/447,753

(22) PCT Filed: Feb. 8, 2007
(86) PCT No.: PCT/CN07/00448

§ 371 Date: Apr. 29, 2009

(30) Foreign Application Priority Data
Nov. 27, 2006 (CN) 200610144965.3

(51) Int. Cl. A61B 5/00

(52) U.S. Cl. 600/323

(57) ABSTRACT

A blood oxygen saturation measuring apparatus comprising an upper shell, a lower shell, and an elastic restoring structure, characterized in that a mark is arranged on a lateral side surface of the upper and/or the lower shell; and the mark is provided to be in alignment with the vertical position of a photoelectric sensing chip which is arranged inside the shell. According to the present invention, the humanized mark is adopted, which can observe the measured position of the measured finger. Even if in the case of weak light environment or a lazy eye user, the accurate and reliable blood oxygen saturation data can be acquired.

### TECHNICAL FIELD

The present invention relates to a fingertip blood oxygen saturation measuring apparatus, in particular, to a fingertip oximeter which can ensure the measuring accuracy.

The term “photoelectric cell” appearing in this specification means all photoelectric sensing elements for measuring the blood oxygen saturation, which can send signals on one side of a measured fingernail and receive the signals on the other side of the fingernail.

### BACKGROUND OF THE INVENTION

At present, the fingertip pulse oximeter available in the market is shown in FIG. 1. The apparatus is of small volume, convenient carrying, and simple use, and is extremely welcomed by people. However, the apparatus has also some drawbacks in use.

For example, an inaccurate displaying or instable displaying of the measured data may occur sometimes during the measurement. The reason is that the measured person's finger positioning in the finger hole is not accurate enough during the measurement. Furthermore, as the thickness and length of the fingers of different measured persons are different, the place of the fingernail may not aim at the photoelectric sensing element in the fingertip pulse oximeter. Because the photoelectric cell in the fingertip pulse oximeter is out of sight from the exterior, the measured person cannot further attempt to aim at the photoelectric cell after the finger is clamped to be measured, which leads to the above mentioned phenomena of inaccurate positioning of the finger.

### SUMMARY OF THE INVENTION

The object of the present invention is to provide a fingertip blood oxygen saturation measuring apparatus, which can accurately measure the blood oxygen saturation in a simple way by setting an exterior mark.

For this purpose, according to one aspect of the present invention, a blood oxygen saturation measuring apparatus is provided, which comprises an upper shell, a lower shell, and an elastic restoring structure, characterized in that a mark is arranged on a lateral side surface of the upper and/or the lower shell, and the mark is provided to be in alignment with the vertical position of a photoelectric sensing chip which is arranged inside the shell.

Preferably, the mark is arranged on the lateral side surface of the shell in a convex or concave manner.

Preferably, a fluorescence mark is used as the mark.

Preferably, a fluorescence mark is used as the mark in the convex or concave manner.

Preferably, the present invention further comprises a detection sensing chip, an internal power source, an internal photoelectric sensing chip, a detecting, processing and controlling circuit, and a display screen.

According to another aspect of the present invention, a blood oxygen saturation measuring probe apparatus is provided, which is connected with a portable oximeter or a monitoring instrument by cables or socket connectors, the oximeter comprises an upper shell, a lower shell, and an elastic restoring structure, characterized in that a mark is provided on a lateral side surface of the upper and/or the lower shell, and the mark is in alignment with the vertical position of a detection photoelectric sensing chip which is arranged inside the shell.

Preferably, the mark is arranged on the lateral side surface of the shell in a convex or concave manner.

Preferably, a fluorescence mark is used as the mark.

Preferably, a fluorescence mark is used as the mark in the convex or concave manner.

According to the present invention, the humanized mark is adopted to observe the measured position of the measured person's finger. Even if in the case of weak light environment or a lazy eye person, the accurate and reliable blood oxygen saturation measured data can be acquired.

### BRIEF DESCRIPTION OF THE DRAWINGS

FIG. 1 is a schematic view of usage of a fingertip pulse oximeter according to the prior art;

FIG. 2 is a perspective view of a fingertip blood oxygen saturation measuring apparatus according to the present invention;

FIG. 3 is a back view of the fingertip blood oxygen saturation measuring apparatus according to the present invention; and

FIG. 4 is a schematic view of a separated blood oxygen saturation measuring probe apparatus according to the present invention.

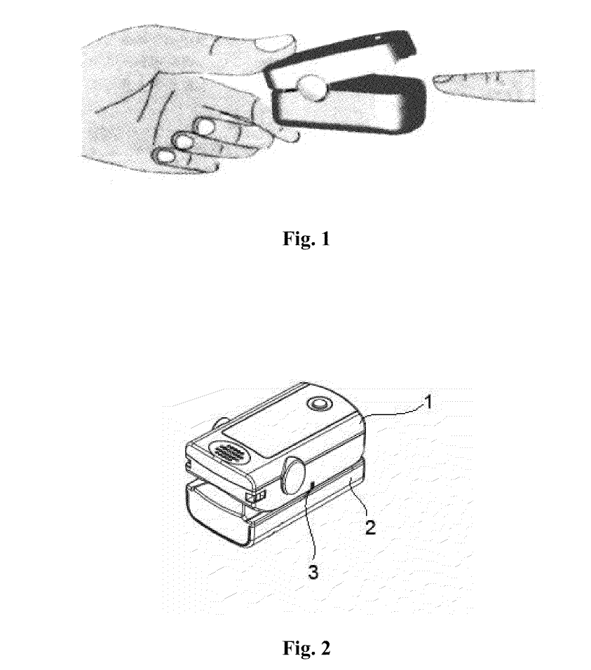

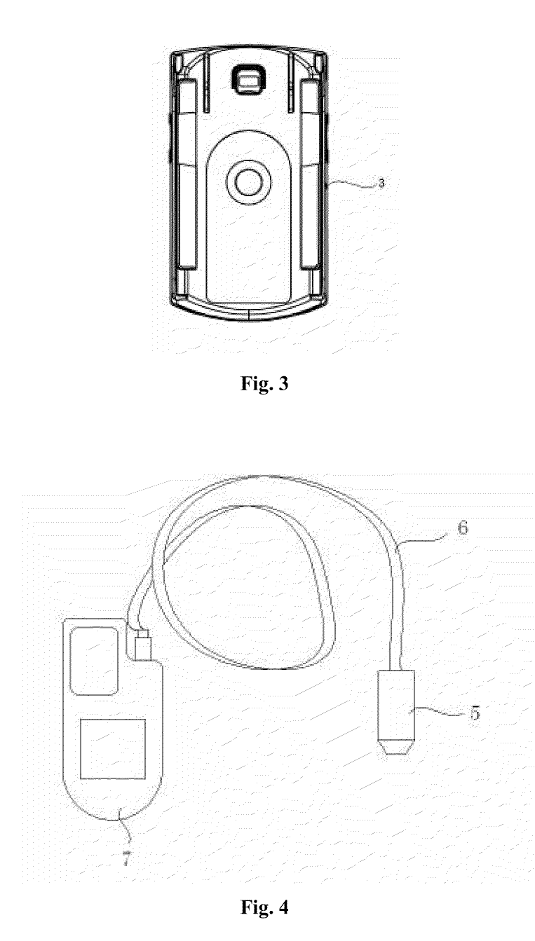

### DETAILED DESCRIPTION OF THE PREFERRED EMBODIMENTS

Next, the embodiments of the present invention are described in more detail in combination with accompanying figures.

As shown in FIG. 2, according to an embodiment of the fingertip blood oxygen saturation measuring apparatus according to the present invention, the fingertip blood oxygen saturation measuring apparatus comprises an upper shell 1, a lower shell 2, an elastic restoring structure, a detection photoelectric sensing chip, an internal power source, an internal photoelectric sensing chip, a detecting, processing and controlling circuit, and a display screen. A mark 3 is arranged on a lateral side surface of the upper shell 1. The vertical line as the mark 3 on the lateral side surface of the upper shell 1 is in alignment with the vertical position of the detection photoelectric sensing chip arranged inside the upper shell 1. The mark 3 is arranged on the lateral side surface of the upper shell 1 in a convex manner. The mark 3 can also be represented its position by fluorescence.

A photoelectric cell A-OE-06 or A-OE-02 can be used as the detection photoelectric sensing chip.

According to the present invention, when the measurement is conducted, the measured person extends his/her one finger into between the upper shell 1 and the lower shell 2. Before the upper shell 1 and the lower shell 2 are loosened from each other, the mark 3 which is arranged on the lateral side surface of the upper shell 1 is made to aim at the fingernail root position of the measured finger. The mark 3 which is arranged on the lateral side surface of the loosened upper shell 1 is rightly aimed at the fingernail root position of the measured finger. At the moment, if the finger positioning has a certain deviation, the finger can be adjusted according to the position of the mark 3 which is arranged on the lateral side surface of the upper shell 1. The accurate measurement can be conducted until the finger positioning is accurate.

As shown in FIG. 4, according to another embodiment of the present invention, the blood oxygen saturation measuring probe apparatus 5 is connected to a prior art portable blood oxygen instrument or a monitoring instrument 7 by cables 6 or socket connectors. With respect to the first embodiment, the main difference of the blood oxygen saturation measuring probe apparatus 5 of the present invention is that the internal power source, the detecting, processing and controlling circuit, as well as the display screen, etc. are arranged inside the portable blood oxygen instrument or the monitoring instrument 7.

The blood oxygen saturation measuring probe apparatus 5 according to the present invention comprises an upper shell 1, a lower shell 2, an elastic restoring structure, and a detection photoelectric sensing chip. A mark 3 is arranged on the lateral side surface of the upper shell 1. The vertical position of the mark 3 on the lateral side surface of the upper shell 1 is provided to be in alignment with the vertical position of the detection photoelectric sensing chip arranged inside the upper shell 1. The mark 3 is arranged on the lateral side surface of the upper shell 1 in a convex manner. The position of the mark 3 can also be represented by fluorescence.

According to another embodiment of the present invention, the mark 3 can also be arranged on the lateral side surface position of the lower shell 2.

According to another embodiment of the present invention, the mark 3 can also be arranged on both the lateral side surface of the upper shell 1 and the lateral side surface of the lower shell 2.

When the measurement is started, the measured person extends his/her one finger into the measuring probe apparatus 5. The mark 3 arranged on the lateral side surface is made to be aimed at the fingernail root position of the measured finger, so as to adjust the position of the finger, and the accurate measurement can be conducted until the finger positioning is accurate.

According to the different demands of the measured persons, the mark 3 which is arranged on the lateral side surface may be convex or concave. Alternatively, the position of the mark 3 can be represented by fluorescence, which satisfies the usage in the case of somewhat dark environment or lazy eye users. Whether the finger positioning is accurate or not can be indicated by fluorescence, or by touching the mark by the other hand, so as to ensure the accurate measurement.

The present invention is described above in detail according to a plurality of embodiments. However, those skilled in the art should understand that various modifications and improvements can be made on the present invention, while such modifications and improvements do not depart from the spirit of the present invention, so the scope of protection will be defined by the attached claims of the present invention.

1. A blood oxygen saturation measuring apparatus comprising an upper shell, a lower shell, and an elastic restoring structure, wherein a mark is arranged on a lateral side surface of the upper and/or the lower shell; and the mark is provided to be in alignment with a vertical position of a photoelectric sensing chip which is arranged inside the shell.

2. The blood oxygen saturation measuring apparatus according to claim 1, wherein the mark is arranged on the lateral side surface of the shell in a convex or concave manner.

3. The blood oxygen saturation measuring apparatus according to claim 1, wherein the mark is a fluorescence mark.

4. The blood oxygen saturation measuring apparatus according to claim 2, wherein the mark in the convex or concave manner is a fluorescence mark.

5. The blood oxygen saturation measuring apparatus according to claim 1, wherein the blood oxygen saturation measuring apparatus further comprises a detection sensing chip, an internal power source, an internal photoelectric sensing chip, a detecting, processing and controlling circuit, and a display screen.
